(19) United States
(12) Patent Application Publication
(10) Pub. No.: US 2017/0105628 A1
(43) Pub. Date: Apr. 20, 2017

(54) PULSE DIAGNOSIS

(71) Applicant: Intel IP Corporation, Santa Clara, CA (US)

(72) Inventors: Jiang Cheng, Xi'an (CN); Xiaolin Yuan, Fremont, CA (US); Jiang Wang, Xi'an (CN); Yiping Tong, Xi'an (CN); Yanzeng Fu, Xi'an (CN)

(73) Assignee: Intel IP Corporation, Santa Clara, CA (US)

(21) Appl. No.: 15/122,564

(22) PCT Filed: Jun. 28, 2014
(86) PCT No.: PCT/CN2014/081069

§ 371 Date: Aug. 30, 2016

(51) Int. Cl. A61B 5/024; A61B 5/16; A61B 5/00

(52) U.S. Cl. CPC A61B 5/024; A61B 5/742; A61B 5/0004; A61B 5/165; A61B 5/681

(57) ABSTRACT

In one example a pulse measurement system, comprises at least one sensor positioned to collect pulse information at three separate locations along a body segment, a controller communicatively coupled to the at least one sensor to receive the pulse information from the at least one sensor, and a display coupled to the controller to present at least one characteristic of the pulse information detected at the three separate locations. Other examples may be described.

### RELATED APPLICATIONS

The present application is a national stage application under 35 U.S.C. §371 of International Application No. PCT/CN2014/081069 filed on Jun. 28, 2014. Said Application No. PCT/CN2014/081069, is hereby incorporated herein by reference in its entirety.

### BACKGROUND

The subject matter described herein relates generally to the field of electronic devices and more particularly to systems and methods for pulse measurement in electronic devices.

Pulse diagnosis a major clinical diagnostic method used in Traditional Chinese medicine (TCM). In TCM pulse diagnosis a TCM doctor palpates six locations, three on each wrist, with the three points called “cun”, “guan”, and “chi”, and describes pulses in terms of various characteristics (FIG. 1). By comparing the pulses at left and right cun, guan, and chi, the health status of individual organs and of the whole body can be determined. As illustrated in FIG. 2, the heart, liver, and kidneys are assessed at the left cun, guan, and chi, respectively, while the lungs, spleen, and kidneys are assessed at the right cun, guan, and chi, respectively. Techniques which enable an electronic device to perform pulse diagnosis may find utility.

### BRIEF DESCRIPTION OF THE DRAWINGS

The detailed description is described with reference to the accompanying figures.

FIG. 1 is a schematic illustration of a technique for pulse diagnosis in accordance with some examples.

FIG. 2 is a schematic illustration of locations for pulse diagnosis accordance with some examples.

FIG. 3 is a schematic illustration of components of an electronic device for pulse diagnosis in accordance with some examples.

FIGS. 4A-4C are schematic illustrations of an electronic device for pulse diagnosis in accordance with some examples.

FIGS. 5A-5B are schematic illustrations of an electronic device for pulse diagnosis in accordance with some examples.

FIGS. 6A-6B are schematic illustrations of an electronic device for pulse diagnosis in accordance with some examples.

FIG. 7 is a schematic illustration of a calibration process for pulse diagnosis in accordance with some examples.

FIG. 8 is a schematic illustration of an architecture health care monitoring and information sharing, in accordance with some examples.

FIG. 9 is a schematic illustration of an architecture for an access management module which may execute on server, in accordance with some examples.

FIG. 10 is a schematic illustration of a data record for pulse data, in accordance with some examples.

FIG. 11 is a schematic illustration of an architecture for remote diagnosis using an electronic devices which may be adapted to pulse diagnosis in accordance with some examples.

FIG. 12 is a schematic illustration of an architecture of a pulse detection module in an electronic devices which may be adapted to pulse diagnosis in accordance with some examples.

FIG. 13 is a schematic illustration of an architecture for sensor module adjustment in an electronic devices which may be adapted to pulse diagnosis in accordance with some examples.

FIG. 14 is a schematic illustration of an architecture for remote diagnosis using an electronic devices which may be adapted to pulse diagnosis in accordance with some examples.

FIG. 15 is a flowchart illustrating operations in a method for pulse diagnosis in accordance with some examples.

FIGS. 16-17 are schematic illustrations of an architecture for remote diagnosis using an electronic devices which may be adapted to pulse diagnosis in accordance with some examples.

FIG. 18 is a flowchart illustrating operations in a method for pulse diagnosis in accordance with some examples.

### DETAILED DESCRIPTION

Described herein are exemplary systems and methods to implement pulse diagnosis in electronic devices. In the following description, numerous specific details are set forth to provide a thorough understanding of various examples. However, it will be understood by those skilled in the art that the various examples may be practiced without the specific details. In other instances, well-known methods, procedures, components, and circuits have not been illustrated or described in detail so as not to obscure the particular examples.

As described above, it may be useful to provide electronic devices with capabilities to perform, or to assist in, pulse diagnosis. based on inputs from sensors in the electronic device. The subject matter described herein addresses these and other issues by providing a pulse diagnosis unit which may be implemented in logic on one or more controller of the electronic device. In some examples, the pulse diagnosis unit utilizes feedback parameters which are adjusted adaptively in response to inputs from an accelerometer sensor and a magnetometer. The feedback parameters may be to adjust sensor characteristics such that the weight of the accelerometer input to the inertial measurement unit decreases as the output of the accelerometer increases. Similarly, the weight of the magnetometer input to the inertial measurement unit decreases as the output of the magnetometer increases. In some examples the weight of the gyroscope input to the inertial measurement unit decreases as the once the location algorithm implemented by the inertial measurement unit has converged.

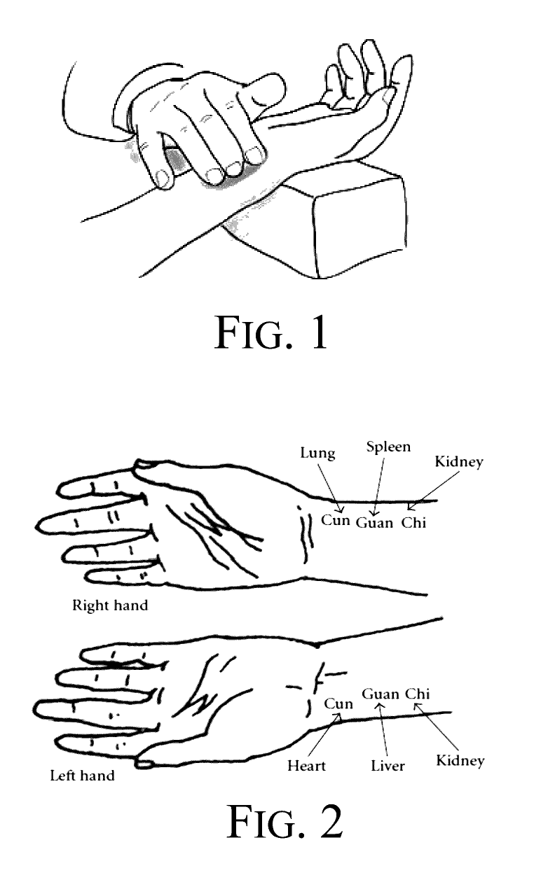

Additional features and operating characteristics of the inertial measurement unit and of electronic devices are described below with reference to FIGS. 1-12.

FIG. 3 is a schematic illustration of components of an electronic device for pulse diagnosis in accordance with some examples. Referring to FIG. 3, in some examples an electronic deice may comprise one or more sensors 310 communicatively coupled to a sensor tuning module 312, which is communicatively coupled to a controller 314. Controller 314 may be coupled to a display 316 and to a communication module 318.

In some examples the sensors 312 may comprise one or more electrodes, along or in combination with another device, to collect pulse information from a subject. Sensor tuning module 312 may comprise logic, at least partially including hardware logic, to tune the sensors 310.

Controller 314 may be embodied as any type of computational element, such as but not limited to, a microprocessor, a microcontroller, a complex instruction set computing (CISC) microprocessor, a reduced instruction set (RISC) microprocessor, a very long instruction word (VLIW) microprocessor, or any other type of processor or processing circuit.

Display 316 may be implemented as a flat panel display or other suitable display device. Communication module 318 may be implemented as a wired interface such as an Ethernet interface (see, e.g., Institute of Electrical and Electronics Engineers/IEEE 802.3-2002) or a wireless interface such as an IEEE 802.11a, b or g-compliant interface (see, e.g., IEEE Standard for IT-Telecommunications and information exchange between systems LAN/MAN—Part II: Wireless LAN Medium Access Control (MAC) and Physical Layer (PHY) specifications Amendment 4: Further Higher Data Rate Extension in the 2.4 GHz Band, 802.11G-2003). Another example of a wireless interface would be a general packet radio service (GPRS) interface (see, e.g., Guidelines on GPRS Handset Requirements, Global System for Mobile Communications/GSM Association, Ver. 3.0.1, December 2002).

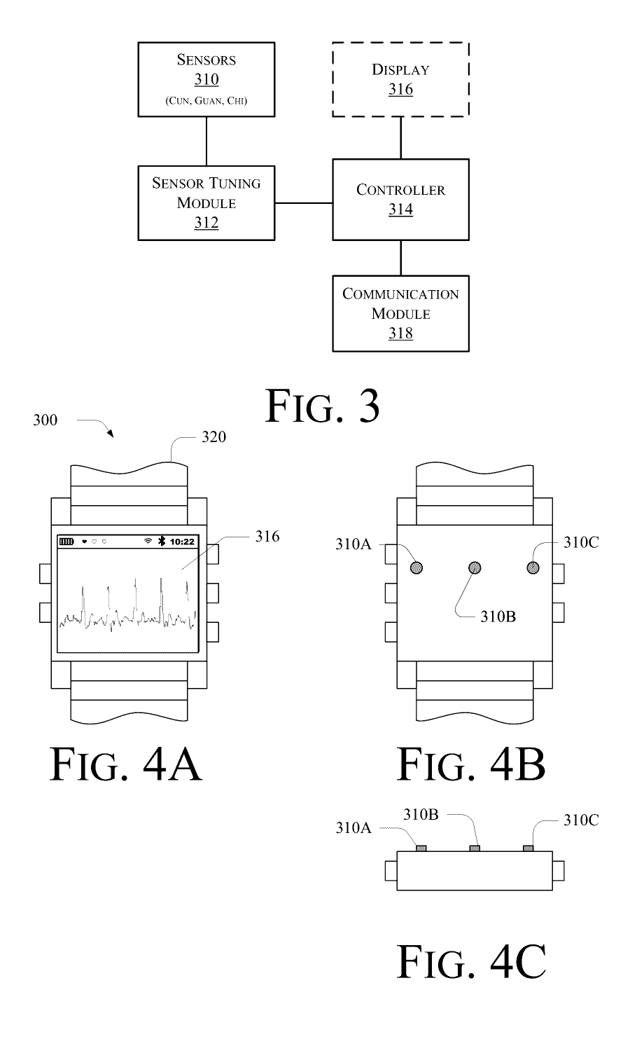

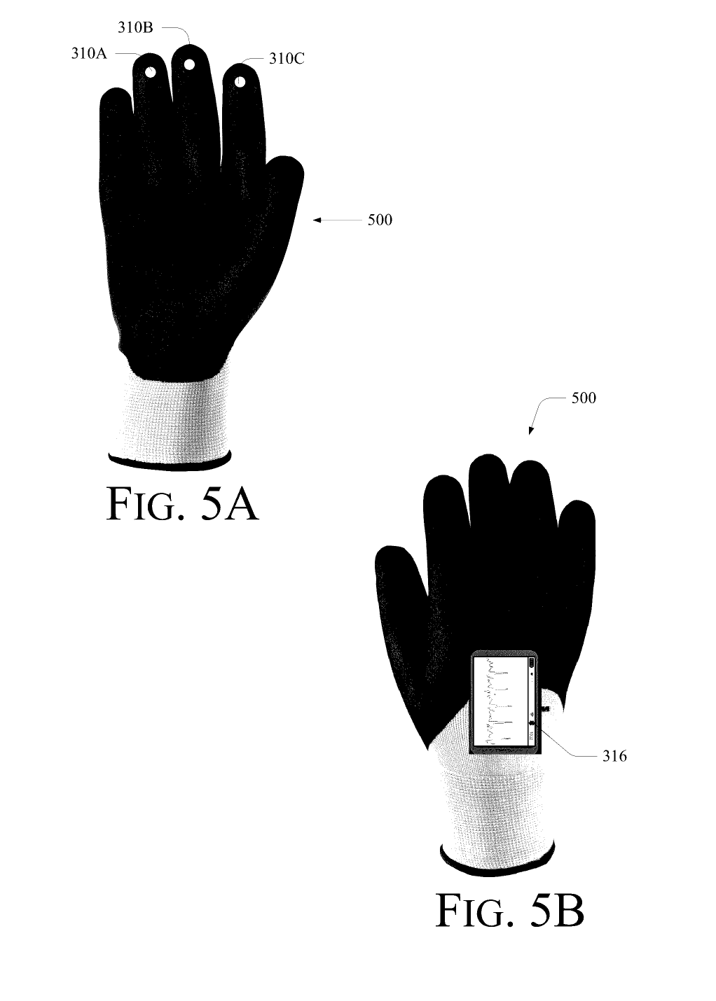

As illustrated in FIGS. 4A-4C, in some examples an electronic device 300 for pulse diagnosis may be implemented as a device capable of being worn about the wrist of a subject. Device 300 may comprise a band 320 and a display 316 mounted on the band 320. Sensors 310A, 310B, 310C may be mounted on a back panel of the display 318 and positioned to collect pulse information from a subject at three separate locations along a body segment, such as the wrist, of the subject. In use, the electronic device 300 may be secured about a wrist of a subject such that the sensors 310A, 310B, 310C are positioned to collect pulse information at three separate locations along the subject's arm. One or more characteristics of the pulse information detected may be presented on display 316.

As illustrated in FIGS. 5A-5B, in some examples an electronic device 300 for pulse diagnosis may be incorporated into a wearable article such as a glove 500. Sensors 310A, 310B, 310C may be embedded into fingertips of the glove 500. Sensors 310A, 310B, 310C may be mounted on a back panel of the display 318 and positioned to collect pulse information from a subject at three separate locations along a body segment, such as the wrist, of the subject. In use, the electronic device 300 may be secured about a wrist of a subject such that the sensors 310A, 310B, 310C are positioned to collect pulse information at three separate locations along the subject's arm. One or more characteristics of the pulse information detected may be presented on display 316.

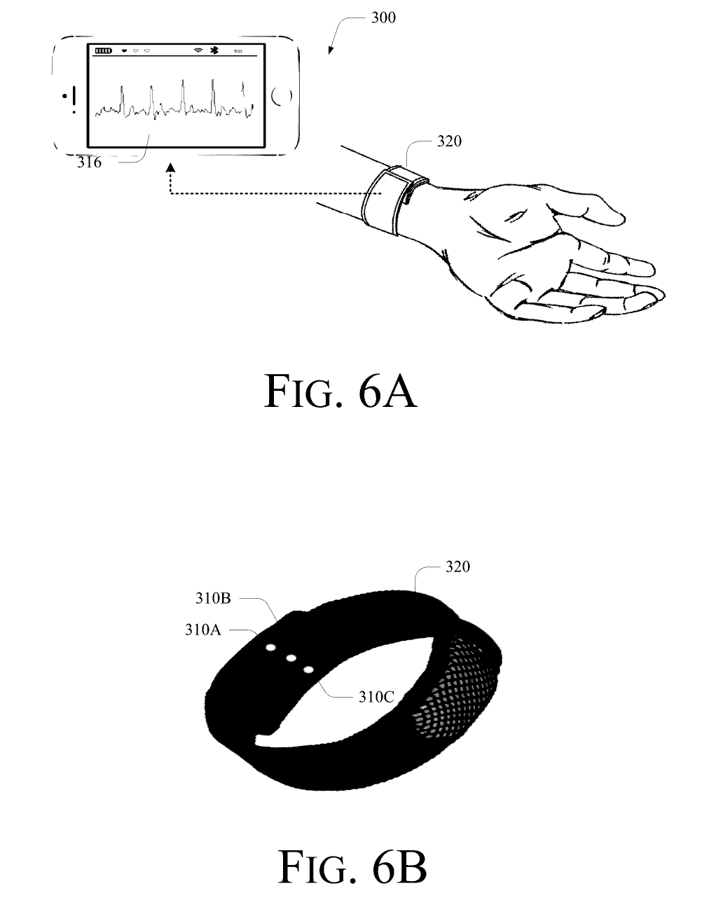

As illustrated in FIGS. 6A-6B, in some examples an electronic device 300 for pulse diagnosis may communicatively coupled to e a band 320 and a display 316 mounted on the band 320. Sensors 310A, 310B, 310C may be mounted on a back panel of the display 318 and positioned to collect pulse information from a subject at three separate locations along a body segment, such as the wrist, of the subject. In use, the electronic device 300 may be secured about a wrist of a subject such that the sensors 310A, 310B, 310C are positioned to collect pulse information at three separate locations along the subject's arm. One or more characteristics of the pulse information detected may be presented on a display 316 of an electronic device 300.

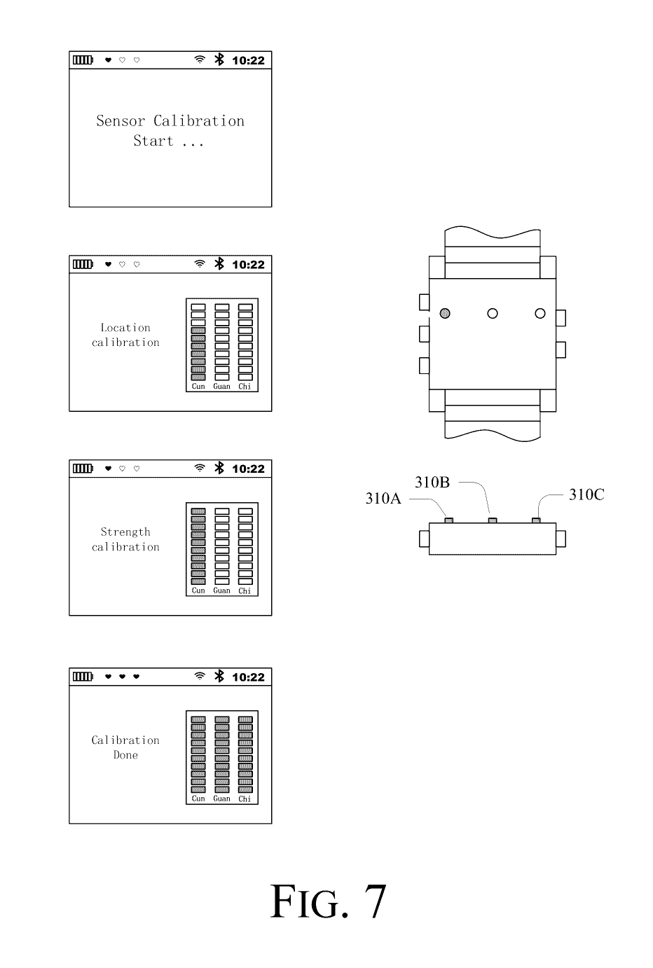

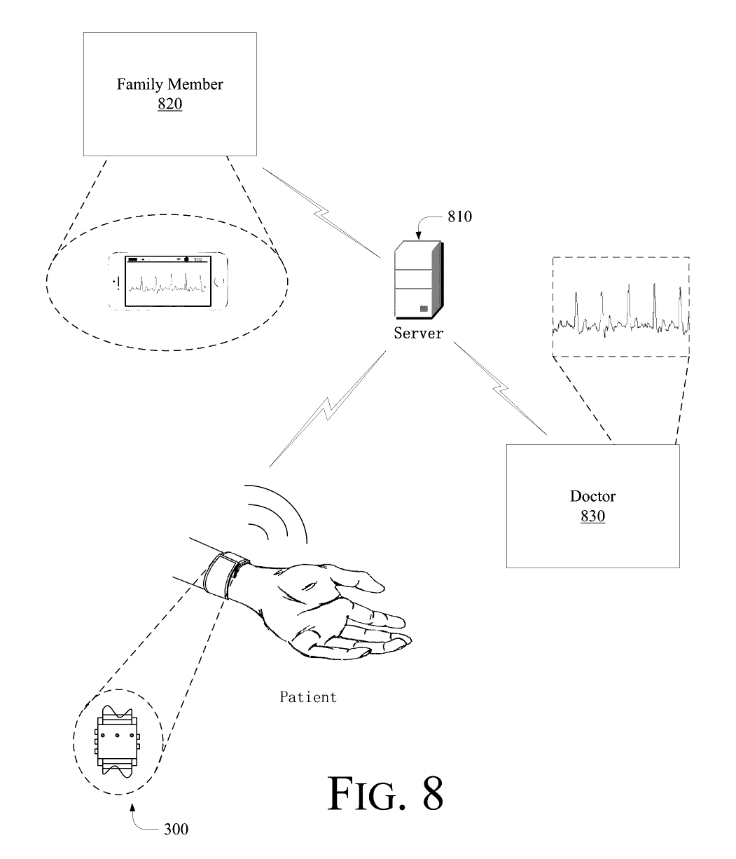

FIG. 7 is a schematic illustration of a calibration process for pulse diagnosis in accordance with some examples. There may be minor differences in the locations of the Cun, Guan and Chi locations on different users and/or differences in the strength of the pulse signal at the respective locations. Thus, in some examples the sensor tuning module 312 may implement a calibration process to calibrate the sensors 310A, 310B, 310C and to adjust the sensor location and/or strength to select a suitable configuration.

In some examples a pulse diagnosis device 300 may be incorporated into a health care monitoring and information sharing system. FIG. 8 is a schematic illustration of an architecture for health care monitoring and information sharing, in accordance with some examples. Referring to FIG. 8, an electronic device for pulse diagnosis 300 may be communicatively couple to a server 810 via a suitable wired or wireless communication connection. Pulse information collected by the pulse diagnosis device 300 may be stored in a computer readable medium, e.g., a database or the like, on server 810. The pulse information may then be accessed by third parties, e.g., a family member 820 or a doctor 830.

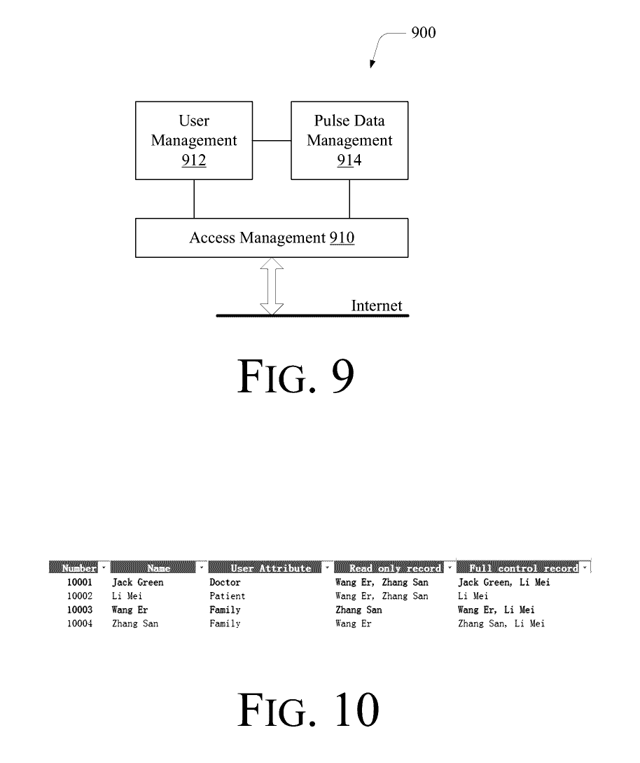

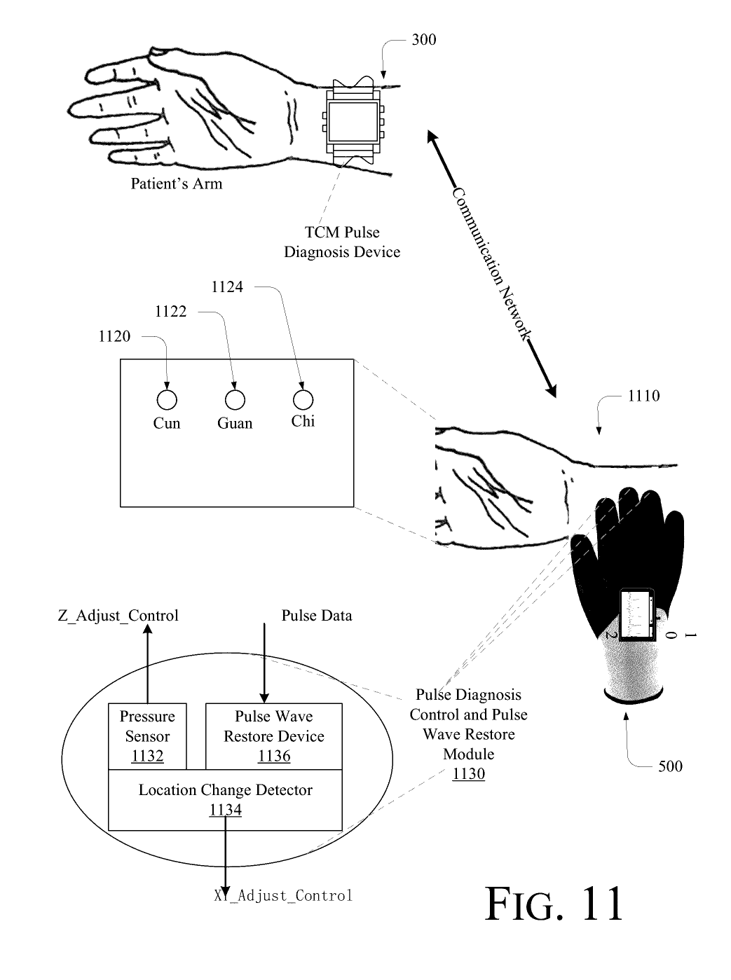

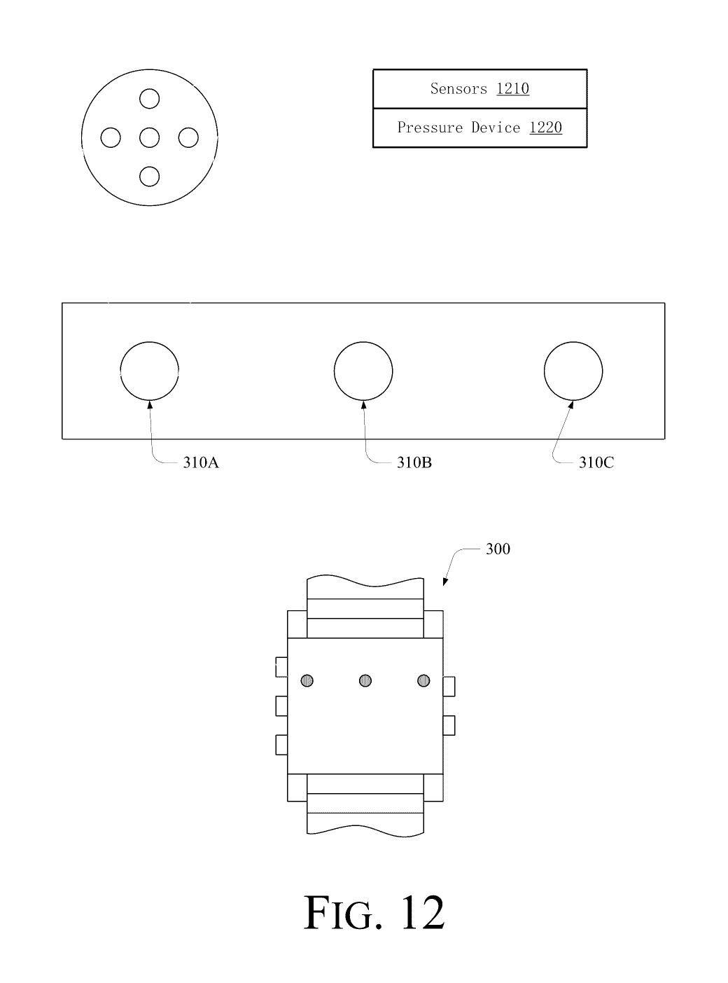

FIG. 9 is a schematic illustration of an architecture for access management which may execute on server 800. Referring to FIG. 9, in some examples an access management module 910 may be communicatively coupled to a pulse diagnosis device 300 via a suitable communication network such as, e.g., the Internet. Access management module 910 may implement logic to regulate who can access data collected from the pulse diagnosis device 300. For example, access management module 910 may require a user to enter a user credential such as user identifier and password combination or other user credentials, to access the data collected from pulse diagnosis device 300.

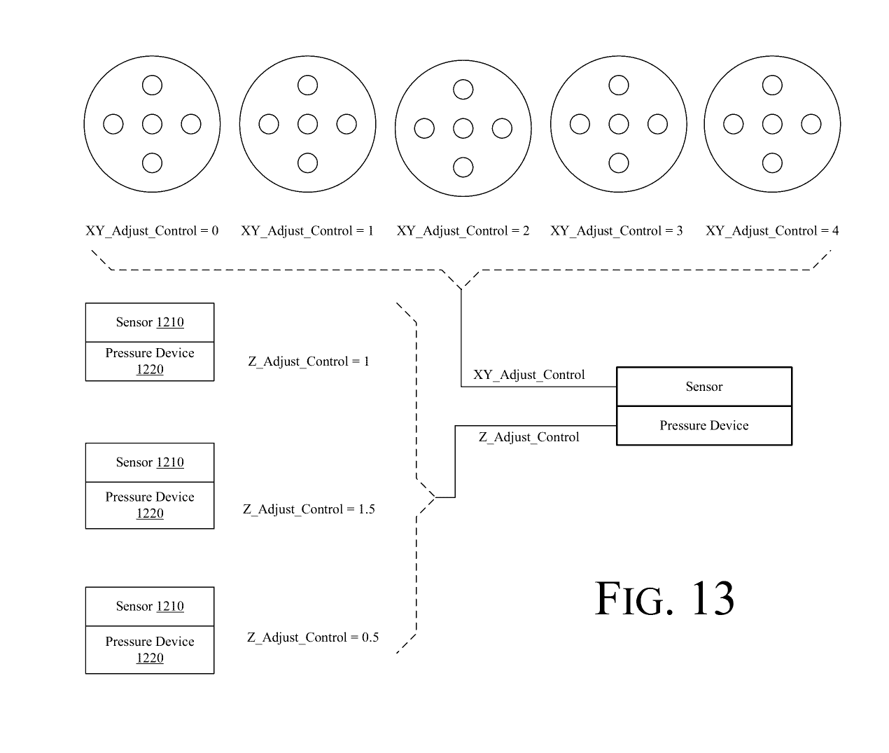

Access management module 910 may be communicatively coupled to a user management module 912 and a pulse data management module 914. User management module 912 may implement logic to manage user access to the system. For example, some users such as doctors may be granted access to the data collected by patients assigned to the doctor, as illustrated in FIG. 10.

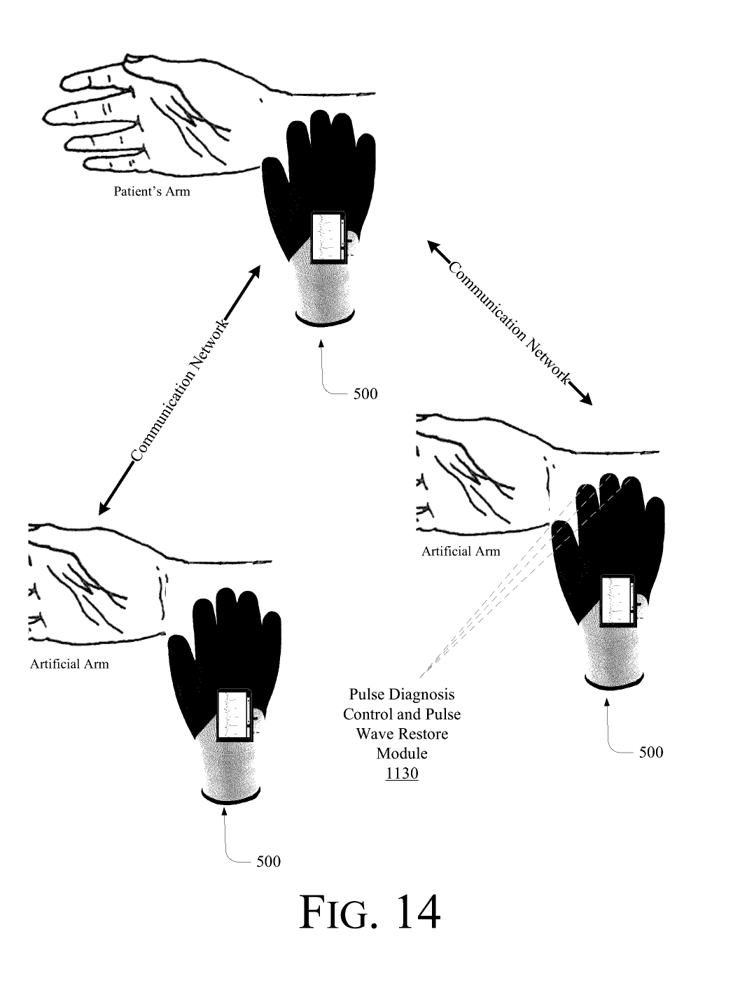

In some examples an electronic device for pulse diagnosis may be incorporated into a system for remote pulse diagnosis. FIG. 11 is a schematic illustration of an architecture for remote diagnosis using an electronic devices which may be adapted to pulse diagnosis in accordance with some examples. Referring to FIG. 11, in some examples the electronic device 300 may be positioned on a subject's wrist. Pulse information collected by the electronic device 300 may be transmitted to a remote device, e.g., an artificial arm 1110 via a suitable communication link.

Artificial arm 1110 may be equipped with three transducers 1120, 1122, 1124 positioned in locations corresponding approximately to the locations of the Cun, Guan, and Chi on the subject's arm. Transducers 1120, 1122, 1124 may be configured to replicate the pulse in the subject's arm which generated the pulse information collected by electronic device 300. A second electronic device, such as a glove 300 as depicted in FIGS. 5A-5B may be positioned to detect pulse information generated by the transducers 1120, 1122, 1124. Electronic device 300 may include a pulse diagnosis control and pulse wave restore module 1130. Said diagnosis control and pulse restore module 1130 includes a pressure sensor 1143 to detect the pressure from the transducers 1120, 1122, 1124, a location change detector 1134 to monitor the movement of the fingers of the glove 300, and a pulse wave restore device 1136 which restore the patient's pulse vibration from received pulse data. The pulse restore device may include a piezoelectric device or magnetic coils which can convert electrical signal to physical movement.

FIG. 12 is a schematic illustration of an architecture of a pulse detection module in an electronic devices which may be adapted to pulse diagnosis in accordance with some examples. Each electronic device 300 includes three sensor modules 310A, 310B, 310C, referred to collectively by reference numeral 310. And for each sensor module 310, there are five (or more) pulse sensors 1210 installed, which gives feasibility to adjust the palpating location. Behinds the sensor(s) 1210 of each sensor module 310, a pressure device 1220 is installed to simulate the palpating strength. The pressure device 1220 may include a mini-inflatable bag and air pump, or other magnetic device can convert electrical signal to physical shape change.

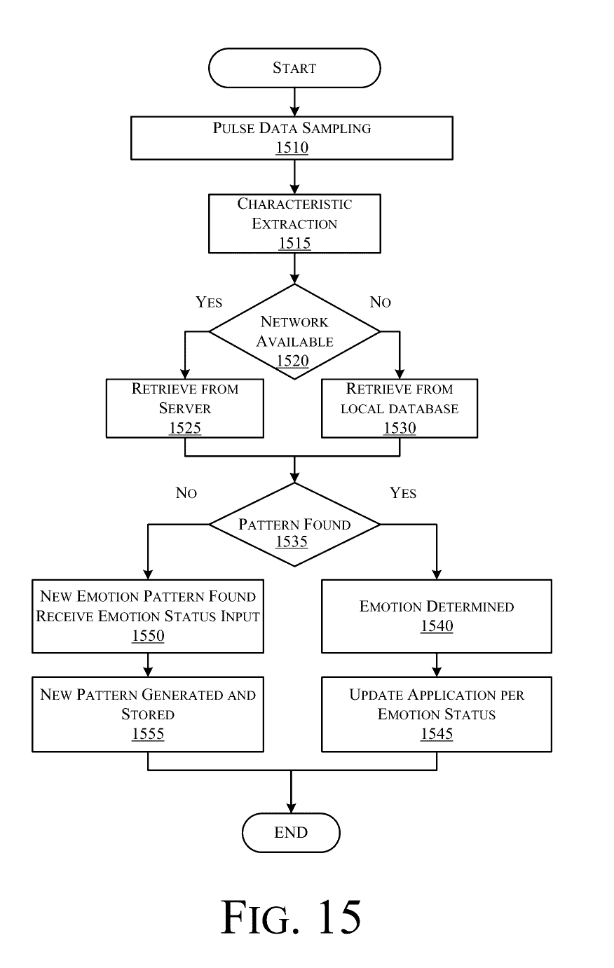

FIG. 13 is a schematic illustration of an architecture for sensor module adjustment in an electronic devices which may be adapted to pulse diagnosis in accordance with some examples. For instance in FIG. 13, each sensor module 310 includes five pulse sensors 1210. An XY_Adjust_Control signal from the glove 500 can select one of the five sensors 1210 for a better palpating accuracy. To simulate the palpating strength the signal received at the glove 500, the pressure device 1220 can adjust its size via a Z_Adjust_Control signal input from the glove 500, so the pressure to patient's wrist changes accordingly. As illustrated in FIG. 14, a signal from a first glove 500 may be replicated in multiple remote gloves 500 to enable the system to be used as a training device.

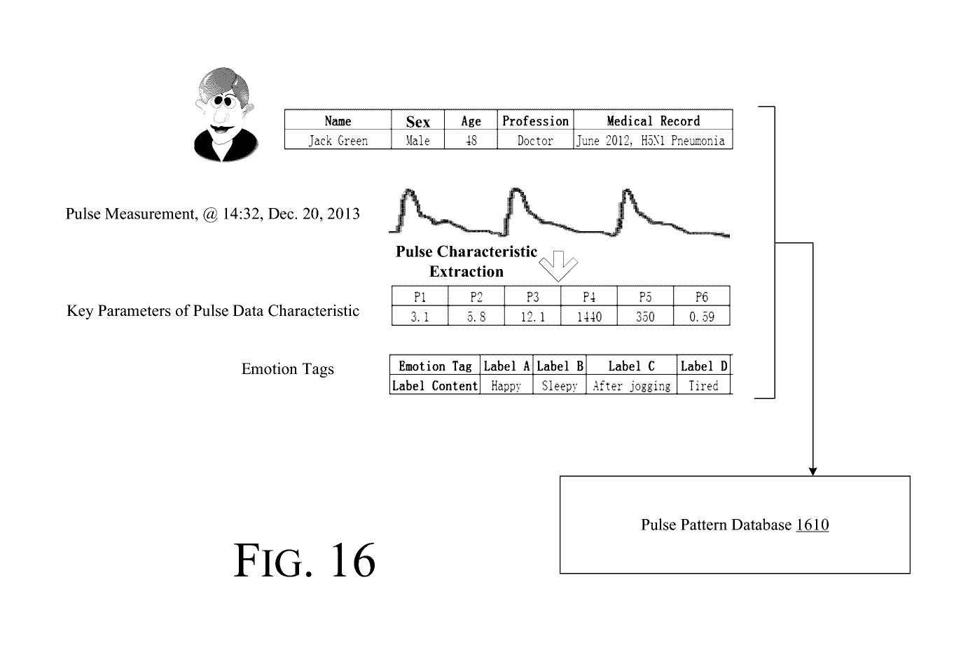

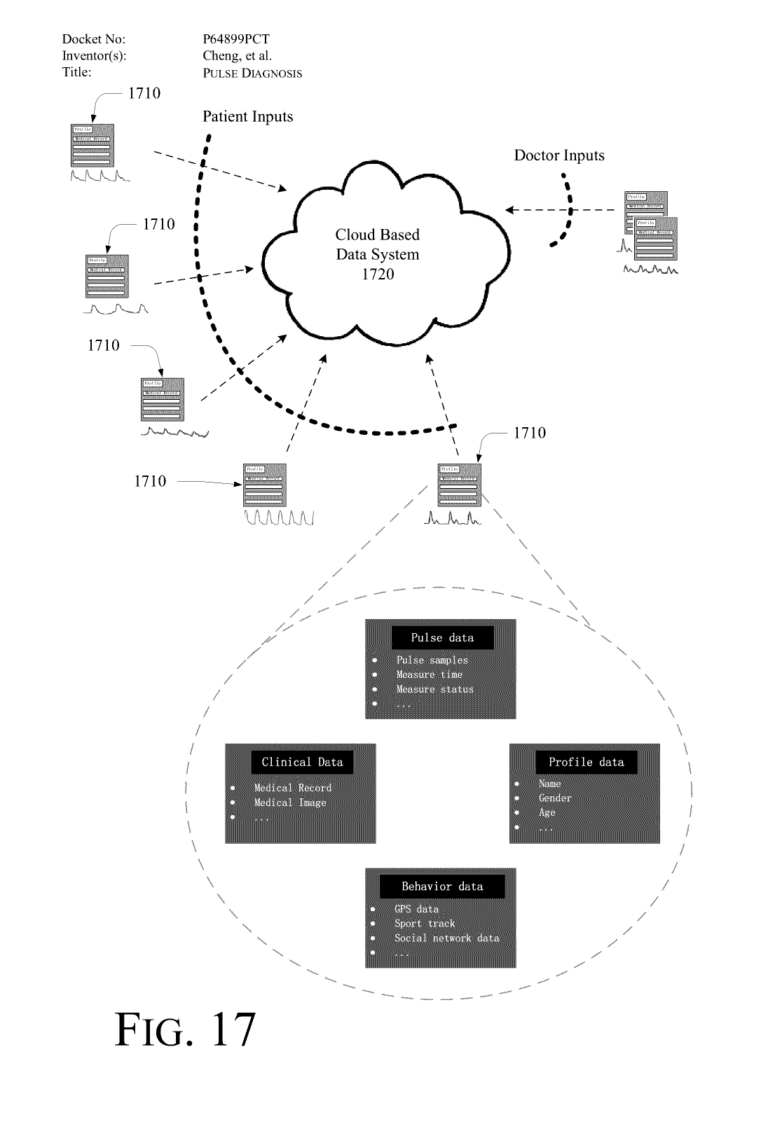

As illustrated in FIGS. 15-17, In some examples the pulse information collected from a patient may be associated with an emotional state of a user and stored in a memory to allow subsequent pulse information from the user to be associated with a user's emotional state. FIG. 15 is a flowchart illustrating operations in a method for pulse diagnosis in accordance with some examples. Referring to FIG. 15, at operation 1510 a pulse sampling operation is initiated, e.g., using one or more of the devices described above. At operation 1515 one or more characteristics of the pulse information collected in operation 1510 are extracted (see FIG. 16).

If, at operation 1520, a network connection is available then control passes to operation 1525 and characteristics are extracted from a remote server. By contrast, if at operation 1520 there is no network connection available then control passes to operation 1530 and characteristics are extracted from a local database 1610 (see FIG. 16).

If, at operation 1535, a pattern is found in the characteristic(s) extracted at operation 1515 that matches the characteristics extracted in operation 1515 then control passes to operation 1540 and the emotional state of the patient is determined by associating the user's emotional state with the emotional state associated with the pattern in memory. At operation 1545 the application is updated to reflect the user's emotional status.

By contrast, if at operation 1535 no pattern is found then control passes to operation 1550 and a new emotional pattern is associated with the characteristic extracted in operation 1515. At operation 1555 the new pattern is generated and stored in memory.

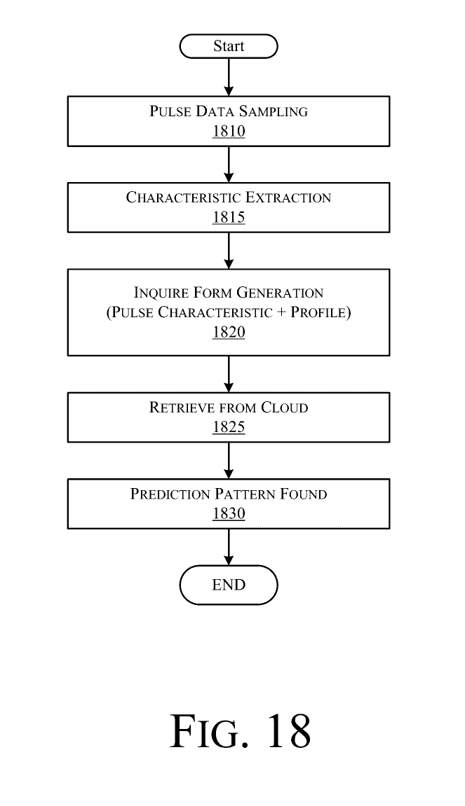

In some examples a pulse diagnosis device may be incorporated into a cloud-based data system, as illustrated in FIGS. 17-18. Referring to FIGS. 17-18, in some examples patient inputs 1710 and doctor inputs 1715 are transmitted to a cloud based data system 1720. The patient data 1710 may include pulse data, clinical data, profile data, and behavior data. The cloud based data system 1720 may receive and process the data, which may be stored in a database.

Subsequently, data collected from patients may be compared against data stored in the cloud based data system for analysis purposes. Referring to FIG. 18, at operation 1810 a pulse data sampling is performed, and at operation 1815 one or more characteristics are extracted from the sampling. At operation 1820 an inquire form is generated, which may include the pulse data characteristic(s) and a patient profile, and is transmitted to the cloud based data system 1720.

At operation 1825 the patient's data is retrieved from the cloud and at 1830 a prediction pattern is found and retrieved from the cloud based data system 1720. The prediction pattern may then be used as a measurement result of a health condition. The following pertains to further examples.

Example 1 is pulse measurement system, comprising at least one sensor positioned to collect pulse information at three separate locations along a body segment, a controller communicatively coupled to the at least one sensor to receive the pulse information from the at least one sensor, and a display coupled to the controller to present at least one characteristic of the pulse information detected at the three separate locations.

In Example 2, the subject matter of Example 1 can optionally include a sensor tuning module communicatively coupled to the at least one sensor and the controller.

In Example 3, the subject matter of any one of Examples 1-2 can optionally include an arrangement in which the sensor tuning module implements a calibration process to adjust the sensitivity of the at least one sensor.

In Example 4, the subject matter of any one of Examples 1-3 can optionally an arrangement in which the at least one sensor is positioned on a surface of a wearable device.

In Example 5, the subject matter of any one of Examples 1-4 can optionally include a communication module coupled to the controller.

In Example 6, the subject matter of any one of Examples 1-5 can optionally include an arrangement in which the communication module provides a communication connection to at least one remote device via a a communication network.

In Example 7, the subject matter of any one of Examples 1-6 can optionally include an arrangement in which the communication module transmits at least a portion of the pulse information to the remote device.

In Example 8, the subject matter of any one of Examples 1-7 can optionally include computing device.

In Example 9, the subject matter of any one of Examples 1-8 can optionally include an arrangement in which the remote device comprises logic, at least partially including hardware logic, to construct a user profile based on the at least a portion of the pulse information transferred to the remote device.

In Example 10, the subject matter of any one of Examples 1-9 can optionally include an arrangement in which the remote device comprises a plurality of actuators configured to actuate in response to the pulse information from the pulse measurement unit.

In Example 11, the subject matter of any one of Examples 1-10 can optionally include an arrangement in which the plurality of actuators are configured to reproduce a physical response that corresponds to the at least one characteristic of the pulse information detected at the three separate locations.

In Example 12, the subject matter of any one of Examples 1-11 can optionally include an arrangement in which a memory coupled to the controller and configured to store the pulse information detected at the three separate locations.

In Example 13, the subject matter of any one of Examples 1-12 can optionally include logic, at least partially including hardware logic, to associate the pulse information stored in the memory with an emotional state.

In Example 14, the subject matter of any one of Examples 1-13 can optionally include logic, at least partially including hardware logic, to generate an output signal corresponding to the emotional state when the pulse information detected at the three separate locations corresponds to the pulse information stored in the memory.

Example 15 is a pulse measurement method, comprising: positioning at least one sensor to collect pulse information at three separate locations along a body segment, receiving the pulse information from the at least one sensor in a controller communicatively coupled to the at least one sensor, and presenting at least one characteristic of the pulse information detected at the three separate locations on a display coupled to the controller.

In Example 16, the subject matter of Example 15 can optionally include a sensor tuning module communicatively coupled to the at least one sensor and the controller.

In Example 17, the subject matter of any one of Examples 15-16 can optionally include an arrangement in which the sensor tuning module implements a calibration process to adjust the sensitivity of the at least one sensor.

In Example 18, the subject matter of any one of Examples 15-17 can optionally an arrangement in which the at least one sensor is positioned on a surface of a wearable device.

In Example 19, the subject matter of any one of Examples 15-18 can optionally include a communication module coupled to the controller.

In Example 20, the subject matter of any one of Examples 15-19 can optionally include an arrangement in which the communication module provides a communication connection to at least one remote device via a a communication network.

In Example 21, the subject matter of any one of Examples 15-20 can optionally include an arrangement in which the communication module transmits at least a portion of the pulse information to the remote device.

In Example 22, the subject matter of any one of Examples 15-21 can optionally include computing device.

In Example 23, the subject matter of any one of Examples 15-22 can optionally include an arrangement in which the remote device comprises logic, at least partially including hardware logic, to construct a user profile based on the at least a portion of the pulse information transferred to the remote device.

In Example 24 the subject matter of any one of Examples 15-23 can optionally include an arrangement in which the remote device comprises a plurality of actuators configured to actuate in response to the pulse information from the pulse measurement unit.

In Example 25, the subject matter of any one of Examples 15-24 can optionally include an arrangement in which the plurality of actuators are configured to reproduce a physical response that corresponds to the at least one characteristic of the pulse information detected at the three separate locations.

In Example 26, the subject matter of any one of Examples 15-25 can optionally include an arrangement in which a memory coupled to the controller and configured to store the pulse information detected at the three separate locations.

In Example 27, the subject matter of any one of Examples 15-26 can optionally include logic, at least partially including hardware logic, to associate the pulse information stored in the memory with an emotional state.

In Example 28, the subject matter of any one of Examples 15-27 can optionally include logic, at least partially including hardware logic, to generate an output signal corresponding to the emotional state when the pulse information detected at the three separate locations corresponds to the pulse information stored in the memory.

The terms “logic instructions” as referred to herein relates to expressions which may be understood by one or more machines for performing one or more logical operations. For example, logic instructions may comprise instructions which are interpretable by a processor compiler for executing one or more operations on one or more data objects. However, this is merely an example of machine-readable instructions and examples are not limited in this respect.

The terms “computer readable medium” as referred to herein relates to media capable of maintaining expressions which are perceivable by one or more machines For example, a computer readable medium may comprise one or more storage devices for storing computer readable instructions or data. Such storage devices may comprise storage media such as, for example, optical, magnetic or semiconductor storage media. However, this is merely an example of a computer readable medium and examples are not limited in this respect.

The term “logic” as referred to herein relates to structure for performing one or more logical operations. For example, logic may comprise circuitry which provides one or more output signals based upon one or more input signals. Such circuitry may comprise a finite state machine which receives a digital input and provides a digital output, or circuitry which provides one or more analog output signals in response to one or more analog input signals. Such circuitry may be provided in an application specific integrated circuit (ASIC) or field programmable gate array (FPGA). Also, logic may comprise machine-readable instructions stored in a memory in combination with processing circuitry to execute such machine-readable instructions. However, these are merely examples of structures which may provide logic and examples are not limited in this respect.

Some of the methods described herein may be embodied as logic instructions on a computer-readable medium. When executed on a processor, the logic instructions cause a processor to be programmed as a special-purpose machine that implements the described methods. The processor, when configured by the logic instructions to execute the methods described herein, constitutes structure for performing the described methods. Alternatively, the methods described herein may be reduced to logic on, e.g., a field programmable gate array (FPGA), an application specific integrated circuit (ASIC) or the like.

In the description and claims, the terms coupled and connected, along with their derivatives, may be used. In particular examples, connected may be used to indicate that two or more elements are in direct physical or electrical contact with each other. Coupled may mean that two or more elements are in direct physical or electrical contact. However, coupled may also mean that two or more elements may not be in direct contact with each other, but yet may still cooperate or interact with each other.

Reference in the specification to “one example” or “some examples” means that a particular feature, structure, or characteristic described in connection with the example is included in at least an implementation. The appearances of the phrase “in one example” in various places in the specification may or may not be all referring to the same example.

Although examples have been described in language specific to structural features and/or methodological acts, it is to be understood that claimed subject matter may not be limited to the specific features or acts described. Rather, the specific features and acts are disclosed as sample forms of implementing the claimed subject matter.

What is claimed is:

1. A pulse measurement system, comprising:
at least one sensor positioned to collect pulse information at three separate locations along a body segment;
a controller communicatively coupled to the at least one sensor to receive the pulse information from the at least one sensor; and
a display coupled to the controller to present at least one characteristic of the pulse information detected at the three separate locations.

2. The pulse measurement system of claim 1, further comprising:
a sensor tuning module communicatively coupled to the at least one sensor and the controller.

3. The pulse measurement system of claim 2 wherein the sensor tuning module implements a calibration process to adjust the sensitivity of the at least one sensor.

4. The pulse measurement system of claim 1, wherein the at least one sensor is positioned on a surface of a wearable device.

5. The pulse measurement system of claim 1, further comprising a communication module coupled to the controller.

6. The pulse measurement system of claim 5, wherein the communication module provides a communication connection to at least one remote device via a a communication network.

7. The pulse measurement system of claim 6, wherein the communication module transmits at least a portion of the pulse information to the remote device.

8. The pulse measurement system of claim 7, wherein the remote device comprises a computing device.

9. The pulse measurement system of claim 8, wherein the remote device comprises logic, at least partially including hardware logic, to:
construct a user profile based on the at least a portion of the pulse information transferred to the remote device.

10. The pulse measurement system of claim 7, wherein the remote device comprises a plurality of actuators configured to actuate in response to the pulse information from the pulse measurement unit.

11. The pulse measurement system of claim 9, wherein the plurality of actuators are configured to reproduce a physical response that corresponds to the at least one characteristic of the pulse information detected at the three separate locations.

12. The pulse measurement system of claim 1, further comprising:
a memory coupled to the controller and configured to store the pulse information detected at the three separate locations.

13. The pulse measurement system of claim 11, further comprising logic, at least partially including hardware logic, to:
associate the pulse information stored in the memory with an emotional state.

14. The pulse measurement system of claim 11, further comprising logic, at least partially including hardware logic, to:
generate an output signal corresponding to the emotional state when the pulse information detected at the three separate locations corresponds to the pulse information stored in the memory.

15. A pulse measurement method, comprising:
positioning at least one sensor to collect pulse information at three separate locations along a body segment;
receiving the pulse information from the at least one sensor in a controller communicatively coupled to the at least one sensor; and
presenting at least one characteristic of the pulse information detected at the three separate locations on a display coupled to the controller.

16. The pulse measurement method of claim 15, further comprising:
a sensor tuning module communicatively coupled to the at least one sensor and the controller.

17. The pulse measurement method of claim 16 wherein the sensor tuning module implements a calibration process to adjust the sensitivity of the at least one sensor.

18. The pulse measurement method of claim 15, wherein the at least one sensor is positioned on a surface of a wearable device.

19. The pulse measurement method of claim 15, further comprising a communication module coupled to the controller.

20. The pulse measurement method of claim 19, wherein the communication module provides a communication connection to at least one remote device via a a communication network.

21. The pulse measurement method of claim 20, wherein the communication module transmits at least a portion of the pulse information to the remote device.

22. The pulse measurement method of claim 21, wherein the remote device comprises a computing device.

23. The pulse measurement method of claim 22, wherein the remote device comprises logic, at least partially including hardware logic, to:
construct a user profile based on the at least a portion of the pulse information transferred to the remote device.

24. The pulse measurement method of claim 22, wherein the remote device comprises a plurality of actuators configured to actuate in response to the pulse information from the pulse measurement unit.

25. The pulse measurement system of claim 24, wherein the plurality of actuators are configured to reproduce a physical response that corresponds to the at least one characteristic of the pulse information detected at the three separate locations.
